(19) United States
(12) Patent Application Publication
(10) Pub. No.: US 2012/0179105 A1
(43) Pub. Date: Jul. 12, 2012

(54) Self-Aligning Abutment

(76) Inventors: Richard A. Schatz, San Diego, CA (US); Christopher J. Birchard, Pasadena, CA (US); Jennifer Maffre, Pasadena, CA (US)

(21) Appl. No.: 13/004,637

(22) Filed: Jan. 11, 2011

(51) Int. Cl. A61M 25/04; B23P 19/00; A61M 25/00

(52) U.S. Cl. 604/175; 604/528; 29/462

(57) ABSTRACT

A catheter system includes a flexible abutment at its distal end. Functionally, the abutment serves to stabilize and align (orient) the distal portion of the catheter as its distal tip is advanced into contact with a tissue surface. Structurally, the flexible abutment is an arrangement of wires that creates a hollow conical shaped frame having two open ends. One end is affixed to the distal end of the catheter. And, the other is presented as a plurality of separated end points that collectively define an annulus at a distal distance from the catheter's tip. For its operation, the flexible abutment is moved from a folded (tube-shaped) configuration, for travel through the vasculature of a patient, to a flared configuration with end points on the frame extended outwardly from the catheter axis, for contact with the tissue surface.

### FIELD OF THE INVENTION

The present invention pertains generally to medical catheters. More specifically, the present invention pertains to catheters that are used for advancing either diagnostic or therapeutic devices into the vasculature of a patient. The present invention is particularly, but not exclusively, useful as a flexible abutment that is mounted on the distal end of a catheter to stabilize and align (orient) the distal portion of a catheter against tissue in the vasculature, during an injection into the tissue.

### BACKGROUND OF THE INVENTION

As a catheter is advanced and positioned in the vasculature of a patient for an operational procedure, control over the catheter is essential. Not surprisingly, it happens that structural aspects of the catheter can significantly contribute to the efficiency and effectiveness of this control. Of particular interest here is the proper positioning of a catheter for the purpose of injecting a fluid medicament into a selected tissue in the vasculature.

To properly inject a fluid medicament into tissue in the vasculature of a patient using an injector catheter, the concern for positioning the catheter is essentially three-fold. Of first concern is the ability to effectively advance the catheter through the vasculature to an appropriate injection site. Next, it is necessary to properly align (i.e. orient) the distal portion of the catheter for the injection. In most instances, such a proper alignment (orientation) requires that the distal portion of the catheter be substantially perpendicular to the surface of the tissue that is to receive the injection. Finally, it is important to prevent a penetration of the tissue by the catheter during an injection procedure. For example, U.S. application Ser. No. 11/422,307 filed on Jun. 5, 2006, for an invention entitled “Myocardial Injector” discusses and discloses such an injection procedure.

In addition to the concerns noted above, it is also desirable to somehow anchor, or stabilize, the distal tip of an injector catheter during an injection procedure. More specifically, once there is contact between the distal tip of the injector catheter and a surface of the tissue that is receiving the injection, it is highly desirable that the distal tip thereafter remain substantially stationary against the surface. As recognized by the present invention, the above-mentioned concerns can be effectively addressed by properly designing a structure for an abutment that can be affixed to the distal end of an injector catheter.

With the above in mind, it is an object of the present invention to provide an injection catheter with a self-aligning distal abutment that stabilizes the injector during an injection procedure. Another object of the present invention is to provide an injection catheter that prevents the catheter from penetrating target tissue during the injection of a fluid medicament into the tissue. Still another object of the present invention is to provide an injection catheter that orients a distal portion of the injector substantially perpendicular to a surface of the target tissue into which a fluid medicament is to be injected. Yet another object of the present invention is to provide an injection catheter that can be manufactured commercially, is easy to use and is comparatively cost effective.

### SUMMARY OF THE INVENTION

In accordance with the present invention, an injector catheter is provided with a flexible abutment that is located at its distal end. Specifically, the structure of this flexible abutment is a wire frame that performs at least two important functions during an operation of the injector catheter. For one, it helps stabilize the distal end (tip) of the injector catheter against the tissue surface where the injection is to be made. For another, the flexible abutment helps align (orient) the distal portion of the catheter so that it will be substantially perpendicular to the tissue surface during the injection.

Structurally, the frame for the flexible abutment of the present invention is attached to a substantially circular end plate. And, the end plate is affixed to the distal end of an injector catheter. For disclosure purposes, the end plate defines a circumference that is centered on a central axis (e.g. the longitudinal axis of the injector catheter). In detail, the flexible abutment (frame) also includes a plurality of elongated tines. Each of the tines has a first end that is pivotally attached to the circumference of the end plate, and it has a second end that is projected outwardly from the end plate. Further, each tine extends perpendicularly from the circumference, and each tine is individually inclined at an inclination angle, (I), relative to the central axis. Also, each tine is equally spaced from its adjacent tines by a substantially same azimuthal angle, θ, that is measured around the central axis. Importantly, the tines are interconnected with each other by a plurality of connectors, and although the inclination angle “φ” may vary somewhat from tine to tine, the azimuthal angle “θ” between adjacent tines will remain substantially constant. Within this structure, the plurality of projected end points (i.e. second ends) of the tines will collectively define an annulus around the central axis.

Also included in the structure of the flexible abutment (frame), is a plurality of connectors. Specifically, these connectors are used to interconnect the individual tines with each other. To do this, each connector is “V” shaped, and it extends between a first base point and a second base point. Each connector also has a flexible apex that is located substantially midway between its two base points. In their relationship with the tines of the flexible abutment, each connector is positioned between a pair of adjacent tines. As so positioned, the first base point of the connector is pivotally affixed to a midpoint on one tine between its first and second ends. Similarly, the second base point of the connector is pivotally affixed to a midpoint between the first and second ends of an adjacent tine.

The flexible aspect of the abutment is due to the various attachments that are established between wires in the frame (i.e. tines and connectors). Specifically, these attachments include pivotal connections between individual tines and the end plate, and pivotal connections between the tines and the base points of respective connectors, as well as the resilience of the apexes of the connectors. When these are all taken together, they give the abutment its overall flexibility. Importantly, although the tines are interconnected as disclosed above, the flexibility of the abutment allows each individual tine to rotate through its own respective angle “φ”. Recall, however, the azimuthal angle “θ” between adjacent tines remains substantially constant. Consequently, a “φ” rotation of each tine will also, at least to some extent, influence the “φ” rotation of its adjacent tines. As envisioned for the present invention, the tines and the connectors are preferably made of nitinol, stainless steel or cobalt chromium.

In a preferred operation of the present invention, the flexible abutment is moveable between a first (folded) configuration and a second (flared) configuration. In the first (folded) configuration, the inclination angle “φ” for each tine is substantially 0°, and each tine is aligned substantially parallel to the central axis. In a preferred embodiment of the present invention, the flexible abutment is held in this first (folded) configuration by being confined within a sheath. Specifically, this first configuration is used while the distal tip of the injector catheter is navigated through the vasculature to the location where it will be used for an injection.

In the second (flared) configuration for the flexible abutment, the inclination angle “φ” is extended into a range between 60° and 80°. To do this, the sheath is merely withdrawn from the flexible abutment. In this second (flared) configuration, the second ends of the respective tines, and to some extent, the apexes of the respective connectors, collectively establish a substantially circular and flexible structure for abutting against an object (e.g. the surface of a tissue).

Functionally, when the flexible abutment is presented in its second (flared) configuration, and the injector catheter is being advanced into contact with tissue for an injection, it will most likely happen that the distal end of the catheter will not be perpendicular to the surface of the tissue. Consequently, only one or two tines of the abutment will make initial contact with a surface of the tissue. The projected end point of these contact tines, however, will become partially embedded into the tissue and will thereby stabilize the catheter relative to the tissue. As the catheter is further advanced, the resultant forces will then be transmitted through the abutment (frame), and the end plate, to cause an alignment of the distal end of the catheter toward a perpendicular orientation relative to the tissue surface. An injection is thus made easier.

### BRIEF DESCRIPTION OF THE DRAWINGS

The novel features of this invention, as well as the invention itself, both as to its structure and its operation, will be best understood from the accompanying drawings, taken in conjunction with the accompanying description, in which similar reference characters refer to similar parts, and in which:

FIG. 1 is a perspective view of a catheter system in accordance with the present invention;

FIG. 2 is a head on view of the distal end (tip) of the catheter, with the flexible abutment of the present invention in its flared configuration;

FIG. 3A is a cross-sectional view of the flexible abutment of the present invention as seen along the line 3-3 in FIG. 1 with the flexible abutment in its folded configuration;

FIG. 3B is a view of the flexible abutment as shown in FIG. 3A, with the flexible abutment in its flared configuration;

FIG. 4A is a side view of the distal portion of a catheter, and the flexible abutment of the present invention, as an initial contact is made by the flexible abutment with a tissue surface in the vasculature of a patient; and

FIG. 4B, in conjunction with FIG. 4A, illustrates the stabilizing and aligning (orienting) capabilities of the flexible abutment as the distal tip of a catheter is advanced into contact with a tissue surface.

### DESCRIPTION OF THE PREFERRED EMBODIMENTS

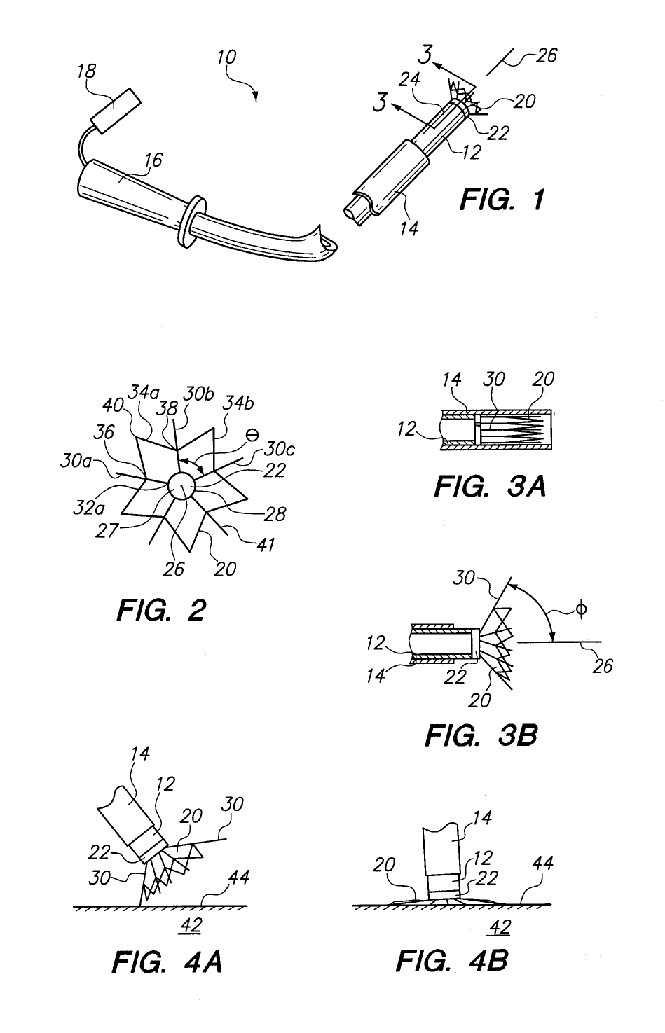

Referring initially to FIG. 1, a catheter system in accordance with the present invention is shown and is generally designated 10. As shown, the system 10 includes a catheter 12, with a sheath 14 that is mounted for reciprocal, longitudinal movement on the catheter 12. FIG. 1 also shows that the system 10 includes a handle 16 and a controller 18 that can be used to move the sheath 14 back and forth on the catheter 12. FIG. 1 also shows that the system 10 includes a frame 20 that is mounted on an end plate 22 at the distal end 24 of the catheter 12.

With reference to FIG. 2, it will be appreciated that the end plate 22 and the catheter 12 define a same central axis 26 that is oriented longitudinally relative to the catheter 12. The end plate 22 preferably has a circular surface 27, and the end plate 22 defines a circumference 28 around the surface 27 of end plate 22 that is centered on the central axis 26. With specific reference now to the frame 20, and as perhaps best seen in FIG. 2, the frame 20 includes a plurality of straight, elongated tines 30. The tines 30a, 30b and 30c are exemplary. Preferably, all of the tines 30 in a frame 20 are arranged around the circumference 28 of end plate 22 with a same azimuthal angle “θ” between adjacent tines 30. Thus, in the case where five tines 30 are used (as shown), the angle θ between adjacent tines 30 will be seventy-two degrees. As also shown, one end of each tine 30 is attached to the circumference 28 of the end plate 22. For the purposes of the present invention, this is a pivotal attachment that allows each of the tines 30 to independently pivot about their respective attachment point 32 (e.g. attachment point 32a for tine 30a). FIG. 2 also shows that located between each pair of adjacent tines 30 (e.g. tine 30a and 30b) there is a “V” shaped connector 34. The connectors 34a and 34b are exemplary.

Using the connector 34a as a specific example, it will be appreciated that the “V” shaped connector 34a has a base point 36 that is pivotally attached to a midpoint on the tine 30a. Also, the connector 34a has a base point 38 that is pivotally attached to a midpoint on the tine 30b. The connector 34a also forms a flexible apex 40 between the base points 36 and 38 of the connector 34a. Importantly, all connections with wire structures in the frame 20 are substantially flexible. To reiterate, each tine 30 of the frame 20 is pivotally attached to the circumference 28 of surface 27 on end plate 22. Also, the respective base points 36 and 38 of each connector 34 are pivotally attached to the midpoint of adjacent tines 30. Further, the apex 40 of each connector 34 is flexible. Consequently, each tine 30 is able to rotate in a plane that includes the particular tine 30 and the central axis 26. And, this rotation will be through an inclination angle “φ” relative to the axis 26 (see FIG. 3B). It will also be appreciated that, although the inclination angle “φ” may differ from one tine 30 to another, the rotation of any tine 30 will influence the rotation of adjacent tines 30, at least to some extent. Consequently, in general, the exposed end points 41 of the respective tines 30 will collectively create an annulus that can expand, contract and tilt. For purposes of the present invention, the tines 30 and connectors 34 are wires that are preferably made of a material such as nitinol, stainless steel or cobalt chromium.

When comparing FIG. 3A with FIG. 3B, it will be appreciated that the frame 20 is moveable between a folded configuration (FIG. 3A) and a flared configuration (FIG. 3B). Specifically, when the sheath 14 is advanced in a distal direction over the frame 20, the frame 20 is folded inside the sheath 14 so that the tines 30 are all aligned substantially parallel to the central axis 26 (i.e. inclination angle φ)=0°). Further, in this folded configuration the tines 30 are protected by the sheath 14. Consequently, the system 10 can be safely advanced into the vasculature of a patient (not shown). On the other hand, when the sheath 14 is withdrawn from the frame 20 in a proximal direction, the constraining effect of the sheath 14 on the frame 20 is removed. As this happens, the frame 20 assumes its flared configuration (i.e. inclination angle φ increases into a range between approximately 60° and 80°).

As intended for the present invention, after the system 10 has been advanced into the vasculature of a patient, and once the distal end 24 of the catheter 12 is located near an intended injection site on tissue 42, the sheath 14 is withdrawn (see FIG. 4A). This action causes the frame 20 to move from its folded configuration (FIG. 3A) to its flared configuration (FIG. 3B). In the event the central axis 26 of the catheter 12 is not reasonably perpendicular to the surface 44 of tissue 42, the end point(s) 41 of only one or two of the tines 30 will initially make contact with the surface 44. The tines 30 that make initial contact with the tissue 42 will then flex and exert a force on the end plate 22 as the distal end 24 of catheter 12 is advanced further toward the tissue 42. This flexing, in turn, allows other tines 30 in the frame 20 to make contact with tissue 42. The consequent collective effect causes the frame 20 to exert a force on the catheter 12 which tends to align the catheter 12 in an orientation that is substantially perpendicular to the surface 44 of the tissue 42. In the specific case where the catheter 12 is an injector catheter, this orientation facilitates the intended injection.

While the particular Self-Aligning Abutment as herein shown and disclosed in detail is fully capable of obtaining the objects and providing the advantages herein before stated, it is to be understood that it is merely illustrative of the presently preferred embodiments of the invention and that no limitations are intended to the details of construction or design herein shown other than as described in the appended claims.

What is claimed is:

1. A flexible abutment for an end plate, wherein the end plate defines a plane and includes a substantially circular surface having a circumference centered on an axis, the abutment comprising:
a plurality of elongated tines, wherein each tine has a first end and a second end with its first end pivotally attached to the circumference of the surface of the end plate, and wherein each tine extends perpendicularly from the circumference and is inclined at a substantially same inclination angle, φ, relative to the axis; and
a plurality of “V” shaped connectors, with each connector extending between a first base point and a second base point with a flexible apex located substantially midway therebetween, wherein each connector is positioned between a pair of adjacent tines with its first base point pivotally affixed to a midpoint on one tine between the first and second ends thereof, and with its second base point pivotally affixed to a midpoint on the adjacent tine between the first and second ends thereof.

2. An abutment as recited in claim 1 wherein the axis is a central axis perpendicular to the circular surface of the end plate, and each tine is equally spaced from adjacent tines with a substantially same azimuthal angle, θ, therebetween, wherein θ is measured around the central axis.

3. An abutment as recited in claim 2 wherein the flexible abutment is moveable between a first configuration wherein the inclination angle φ is substantially 0°, with each tine being substantially parallel to the central axis, and a second configuration wherein the inclination angle φ is in a range between 60° and 80°.

4. An abutment as recited in claim 1 wherein the second ends of the respective tines and the apexes of the respective connectors collectively establish a substantially circular and flexible structure for abutting against an object.

5. An abutment as recited in claim 4 wherein the object is tissue in the vasculature of a patient.

6. An abutment as recited in claim 1 wherein the end plate is affixed to a distal end of a catheter.

7. An abutment as recited in claim 1 wherein the tines and the connectors are made of a material selected from a group comprising nitinol, stainless steel and cobalt chromium.

8. A catheter system which comprises:
a base catheter having a proximal end and a distal end, and defining an axis;
an end plate mounted on the base catheter at the distal end thereof, wherein the end plate defines a plane and includes a substantially circular surface having a circumference centered on the axis of the base catheter;
a flexible frame mounted on the end plate, with the frame being affixed to the end plate along the circumference of the circular surface to present a plurality of end points projecting in a distal distance from the end plate, with the plurality of end points collectively defining an annulus around the axis; and
a sheath mounted for sliding movement on the base catheter between a distal position wherein the sheath encloses the flexible frame to place the frame in a folded configuration where its end points are juxtaposed with the axis of the base catheter, and a proximal position wherein the sheath has been withdrawn from the flexible frame to place the frame in a flared configuration with all of its end points radially distanced from the axis of the base catheter to establish a substantially circular and flexible structure for abutting against an object.

9. A catheter system as recited in claim 8 wherein the flexible frame comprises:
a plurality of elongated tines, wherein each tine has a first end and a second end with its first end pivotally attached to the circumference of the surface of the end plate, and wherein each tine extends perpendicularly from the circumference and is inclined at a substantially same inclination angle, φ, relative to the axis; and
a plurality of “V” shaped connectors, with each connector extending between a first base point and a second base point with a flexible apex located substantially midway therebetween, wherein each connector is positioned between a pair of adjacent tines with its first base point pivotally affixed to a midpoint on one tine between the first and second ends thereof, and with its second base point pivotally affixed to a midpoint on the adjacent tine between the first and second ends thereof.

10. A catheter system as recited in claim 9 wherein the respective second ends of the tines and the respective apexes of the connectors are the plurality of end points of the flexible frame.

11. A catheter system as recited in claim 9 wherein each tine is equally spaced from adjacent tines with a substantially same azimuthal angle, θ, therebetween, wherein θ is measured around the axis.

12. A catheter system as recited in claim 9 wherein the flexible frame is moveable between a first configuration wherein the inclination angle φ is 90° with each tine being substantially perpendicular to the surface and parallel to the central axis, and a second configuration wherein the inclination angle φ is in a range between 60° and 80°.

13. A catheter system as recited in claim 9 wherein the second ends of the respective tines and the apexes of the respective connectors collectively establish a substantially circular and flexible structure for abutting against an object.

14. A catheter system as recited in claim 13 wherein the object is tissue in the vasculature of a patient.

15. A catheter system as recited in claim 8 wherein the sheath is a guide catheter, and wherein the tines and the connectors are made of a material selected from a group comprising nitinol, stainless steel and cobalt chromium.

16. A method for manufacturing a catheter system which comprises the steps of:
providing an end plate, wherein the end plate defines a plane and includes a substantially flat circular surface having a circumference centered on an axis;
affixing a plurality of tines to the circumference of the end plate, wherein each tine has a first end and a second end, and wherein each tine extends perpendicularly from the circumference;
inclining each tine at a substantially same inclination angle, (I), relative to the axis; and
interconnecting the plurality of tines with a plurality of “V” shaped connectors to create a flexible frame, wherein each connector extends between a first base point and a second base point with a flexible apex located substantially midway therebetween, wherein each connector is positioned between a pair of adjacent tines with its first base point being pivotally connected to a midpoint on one tine between the first and second ends thereof, and with its second base point being pivotally connected to a midpoint on the adjacent tine between the first and second ends thereof.

17. A method as recited in claim 16 wherein the circular surface defines a central axis perpendicular thereto and the affixing step includes the steps of:
locating the first end of each tine on the circumference of the surface of the end plate; and
joining the first end of each tine to the end plate to establish a pivotal attachment therebetween.

18. A method as recited in claim 17 wherein the locating step is accomplished by spacing each tine from adjacent tines with a substantially same azimuthal angle, θ, therebetween, wherein θ is measured around the central axis.

19. A method as recited in claim 16 further comprising the steps of:
mounting the end plate at a distal end of a catheter; and
inserting the catheter into a sheath, wherein the sheath is moveable on the catheter between a distal position wherein the sheath encloses the flexible frame to place the frame in a folded configuration where its end points are juxtaposed with the central axis, and a proximal position wherein the sheath has been withdrawn from the flexible frame to place the frame in a flared configuration with all of its end points radially distanced from the axis of the base catheter to establish a substantially circular and flexible structure for abutting against an object.

20. A method as recited in claim 19 wherein the object is tissue in the vasculature of a patient.
